(19) United States
(12) Patent Application Publication
(10) Pub. No.: US 2003/0091526 A1
(43) Pub. Date: May 15, 2003

(54) Treatment of hair by addition of exogenous enzymes, alcohols and acids or triglycerides

(76) Inventors: Genevieve Kaba, Ivry s/Seine (FR); Didier Semeiria, Livry-Gargan (FR); Yolanda Duvault, Aulnay s/Bois (FR)

Correspondence Address: NIXON & VANDERHYE, PC, 1100 N GLEBE ROAD, 8TH FLOOR, ARLINGTON, VA, 22201-4714, US

(21) Appl. No.: 10/265,901

(22) Filed: Oct. 8, 2002

(30) Foreign Application Priority Data
Nov. 10, 2001 (FR) 0113112
Nov. 10, 2001 (FR) 0113113

(51) Int. Cl. A61K 7/06; A61K 7/11; C12P 7/64; C12P 7/62

(52) U.S. Cl. 424/70.14; 424/401; 435/135; 435/134

(57) ABSTRACT

The present invention relates to compositions for treating keratin materials by adding at least one exogenous enzyme, long-chain alcohols, and long-chain acids or triglycerides, which gives rise to an enzymatic reaction directly on the hair, and to methods of treating hair which employ these compositions.

The present invention relates to compositions for treating keratin materials by adding at least one exogenous enzyme, long-chain alcohols, and long-chain acids or triglycerides, which gives rise to an enzymatic reaction directly on the hair, and to methods of treating hair which employ these compositions.

The hair is subject to continual aggressive influences, which may make it brittle, difficult to disentangle or dull. It is therefore important to find products capable of conditioning the hair and so leaving it easy to style, supple, soft to the touch, and lustrous. It would also be advantageous for the properties of softness and sheen of these products to persist over a number of shampooings.

It has been observed that the soft and shiny appearance of the treated fibre is particularly great if long-chain esters are applied to the hair.

Proposals have already been made to use enzymes as conditioning agents for the hair. Such compositions are described in particular in the patent application WO 00/64405, in which the composition which enhances the appearance of the hair requires the presence of an endogenous enzyme which is present in the hair fibre.

The patent application DE 1 982 474 describes the use of exogenous enzymes with the aim of reversing the process of sebum hydrolysis through reesterification of the glycerol and of the sebum fatty acids which are generated in situ.

Other hair treatment compositions whose method involves an enzymatic reaction have been described, particularly in the patent applications EP 293 0 01, JP 081 173 787 and EP 391 431.

The applicant has found that the in situ formation of fatty esters by combining long-chain acids or triglycerides, long-chain alcohols and at least one or more exogenous enzymes which are capable of bringing about an esterification reaction enhances the soft and shiny appearance of the hair fibre thus treated, makes it easy to style, and imparts to it manageability.

The applicant has further found, surprisingly, that when the esterification or transesterification reaction is carried out in situ directly on the hair the result for the keratin materials in terms of sheen and softness is greater than simply depositing the corresponding esters synthesized on their own. Furthermore, these properties are conserved after a number of shampooings.

The invention additionally provides a method of treating hair which employs these compositions.

Further subject-matter of the present application will emerge upon reading the description and examples which follow.

The cosmetic compositions of the invention comprise at least one alcohol of formula R₁—OH, in which R₁ is an optionally substituted, saturated or unsaturated, linear or branched C₃ to C₃₂ alkyl group with or without polyethoxylation, at least one acid of general formula R₂—COOH, in which R₂ is an optionally substituted, saturated or unsaturated, linear or branched C₃ to C₃₁ alkyl group, with the number of carbon atoms in (R₁+R₂) 8, or at least one triglyceride, and at least one exogenous enzyme capable of inducing an esterification reaction.

The cosmetic compositions of the invention comprise at least one alcohol of formula R₁—OH, in which R₁ is an optionally substituted, saturated or unsaturated, linear or branched C₃ to C₃₂ alkyl group with or without polyethoxylation, at least one acid of general formula R₂—COOH, in which R₂ is an optionally substituted, saturated or unsaturated, linear or branched C₃ to C₃₁ alkyl group, with the number of carbon atoms in (R₁+R₂) 8, and at least one enzyme capable of inducing an esterification reaction.

The cosmetic compositions of the invention comprise at least one alcohol of formula R₁—OH, in which R₁ is an optionally substituted, saturated or unsaturated, linear or branched C₃ to C₃₂ alkyl group with or without polyethoxylation, at least one triglyceride, and at least one exogenous enzyme capable of inducing an esterification reaction.

The alcohols which may be used in accordance with the invention are selected in particular from the following alcohols: hexanol, 2-ethylhexanol, octanol, 2-butyl-octanol, nonanol, decanol, 2-hexyldecanol, undecanol, dodecanol, 2-octyidodecanol, tridecanol, tetradecanol, 2-decyltetradecanol, pentadecanol, hexadecanol, 2-dodecylhexadecanol, octadecanol, 2-tetradecyl-octadecanol, nonadecanol, tricosanol, hexacosanol, triacontanol, arachinyl, behenyl, lignoceryl, palmitoleyl, oleyl, elaidyl, ricinoleyl, linoleyl, linolenyl, arachidonyl and erucyl alcohols, polyethoxylated alcohols such as polyoxyethylene lauryl ether, polyoxyethylene cetyl ether and polyoxyethylene stearyl ether, and the derivatives sold under the name “Brij” by the company ICI.

The acids which can be used in accordance with the invention are selected in particular from the following acids: hexanoic, heptanoic, octanoic, pelargonic, decanoic, undecanoic, dodecanoic, tetradecanoic, hexadecanoic, octadecanoic, eicosanoic, docosanoic, tetracosanoic, hexacosanoic, triacontanoic, undecenoic, palmitic, palmitoleic, oleic, elaidic, linoleic, linolenic, ricinoleic, arachidonic, erucic, brassidic, nervonic, stearic, 12-hydroxystearic, isostearic, 2-butyloctanoic, 2-hexyldecanoic, 2-decylmyristic, 2-laurylhexadecanoic, 2-tetradecyloctadecanoic, 2-pentadecylnonadecanoic, 2-hexadecyleicosanoic and 2-octyidodecanoic acids.

The triglycerides which can be used in accordance with the invention are selected in particular from the following triglycerides: glycerol trihexanoate, glycerol tricaprylate, glycerol trinonanoate, glycerol tridecanoate, glycerol triundecanoate, glycerol tri-laurate, glycerol tripentadecanoate, glycerol trimy-ristate, glycerol tridecanoate, glycerol tripalmitate, glycerol tristearate, glycerol triarachidonate, gly-cerol tribehenate, glycerol tri(cis-9-tetradecenoate), glycerol tri(cis-9-hexadecenoate), glycerol tri(transhexadecenoate), glycerol trioleate, glycerol tri(trans-9-octadecenoate), glycerol tri(cis,cis-9,12-octadeca-dienoate), glycerol tri(cis,cis,cis,cis-5,8,11,14-eico-satetraenoate), glycerol tri(cis-13-docosenoate), glycerol tri(cis-15-tetracosenoate), and triglycerides of coprah oil, palm oil, soya oil, jojoba oil, colza oil, palm kernel oil, almond oil, castor oil, sunflower oil, safflower oil, borage oil, and corn germ oil, callow triglycerides, cocoa butter and karite butter.

These products are marketed in particular by the company Sigma.

The enzymes capable of inducing esterification are selected in particular from lipases, esterases and proteases. They may be either free or immobilized on various supports.

As proteases in accordance with the invention mention may be made, inter alia, of proteases of animal origin (pepsin, chymotrypsin, trypsin, rennin, pancreatin, collagenase, elastase, etc.), proteases of plant origin (papain, chymopapain, bromelain, ficin, etc.) or proteases of microbial origin (of fungal, bacterial or yeast origin, such as Aspergillus, Penicillium, Rhizopus or Mucor, etc.); these various proteases are marketed by the companies Amano, Novo, Sigma, Boehringer, Nagase, Green Cross Corp., etc.

As lipases according to the invention mention may be made, inter alia, of lipases of animal origin (pancreatic lipase, lipase from milk, etc.), lipases of plant or microbial (fungal, bacterial or yeast) origin such as the fungal lipases from Mucor miehi, Aspergillus niger, Rhizopus arrhizus, Rhizopus delemar, Rhizopus japonicus, Rhizopus oryzae, Rhizopus sp., Geotrichum candidum, etc., the bacterial lipases from Chromobacterium viscosum, Arthrobacter ureafaciens, Achromobacter sp., Pseudomonas aeruginosa, Pseudomonas fluorescens, Pseudomonas sp., Alcaligenes sp., etc., the yeast lipases from Candida cylindracea, Candida rugosa, Candida lipolytica, Candida antarctica, etc. These various lipases are marketed by the companies Amano, Novo, Sigma, Fluka, Biocatalysts, Godo Shusei, Meito sangyo, etc.

As esterases in accordance with the invention mention may be made, inter alia, of pig liver esterases, rabbit liver esterases and pancreatic esterases, which are marketed by the company Sigma.

A large number of industrial enzymes useful to the invention are set out in the book “Industrial enzymes and their applications” by Helmuth UHLIG, 1998, John WILEY Inc., New York. Enzymes may where appropriate also be obtained from genetic recombinations (recombinant enzymes).

When these enzymes are used in support-immobilized form, a very large number of supports are possible, such as, inter alia, resins, the diatomaceous earth with the brand name Celite, Nylon, porous glass, cellulose, polyethylene glycol, etc., and are set out in the Journal of the American Oil Chemists' Society, 1990, Vol. 67, No. 12, pp. 890-910; among these it is possible to retain the Lipozymes RM IM, IM 60, and Novozym 435 from NOVO Nordisk.

The amount of enzymes present in the cosmetic composition of the invention is between 0.0001 and 50% by weight relative to the weight of the mixture by weight of acids and alcohols or of triglycerides and alcohols. The amount of enzymes is preferably between 0.1 and 20% by weight relative to the weight of the mixture by weight of acids and alcohols or of triglycerides and alcohols.

In the cosmetic composition of the invention the proportion of acids may vary between 1 and 99% relative to the mixture by weight of acids and alcohols. Preferably the proportion of acids may vary between 30 and 70% relative to the mixture by weight of acids and alcohols.

In the cosmetic composition of the invention the proportion of triglycerides may vary between 1 and 99% relative to the mixture by weight of alcohols and triglycerides. Preferably the proportion of triglycerides may vary between 30 and 70% relative to the mixture by weight of alcohols and triglycerides.

In the cosmetic composition of the invention the proportion of alcohols may vary between 1 and 99% relative to the mixture by weight of acids and alcohols or of triglycerides and alcohols.

Preferably the proportion of alcohols may vary between 30 and 70% relative to the mixture by weight of acids and alcohols or of triglycerides and alcohols.

Besides the abovementioned mandatory constituents, the composition of the invention may comprise adjuvants selected from carbonic oils, silicone oils, fluoro oils, gelling agents, thickeners, emollients, softeners, antioxidants, opacifiers, stabilizers, ionic, non-ionic or amphoteric polymers, vitamins, perfumes, preservatives, dyes, pigments or any other adjuvant which is commonly used in cosmetology and is compatible with the enzyme or enzymes that are used.

The cosmetic composition of the invention is in the form of lotions, gels or creams intended for rinsing or otherwise.

The invention further provides a device comprising a plurality of compartments (at least two) each containing one or two of the following components:

one or more alcohols of formula R₁—OH in which R₁ is an optionally substituted, saturated or unsaturated, linear or branched C₃ to C₃₂ alkyl group with or without polyethoxylation,

one or more acids of general formula R₂—COOH in which R₂ is an optionally substituted, saturated or unsaturated, linear or branched C₃ to C₃₁ alkyl group, with the number of carbons in (R₁+R₂) 8, or one or more triglycerides,

one or more exogenous enzymes capable of inducing esterification, the entirety of the device comprising at least one acid, an alcohol or a triglyceride and an enzyme. The various components of the device are mixed immediately prior to use.

The invention further provides for the use of the compositions of the invention for imparting a soft, shiny and easy-to-style appearance to the hair fibre.

The invention additionally provides a method of cosmetically treating the hair to impart a soft and shiny appearance to the fibre. The way in which the cosmetic compositions of the invention are used is as follows: the acids, the alcohols or the triglycerides and the enzymes are mixed immediately prior to use (between 1 second and 5 minutes) and the resulting composition is applied immediately to the keratin fibres, is left to act for from 1 to 60 minutes at a temperature between 20 and 65° C., preferably between 30 and 65° C., and is rinsed with water where appropriate.

A further subject of the invention is that the treatment method involves an esterification or transesterification reaction which takes place in situ on the hair.

The examples which follow are intended to illustrate the invention without, however, exhibiting any limitative character.

### EXAMPLE 1

Remanence Measured for an Acid/alcohol/enzyme Reaction Effected in situ on the Hair

The locks of hair used are selected for the quality of the chromatographed sebum.

In a 200 ml wide-necked flask a mixture is formed from

380 mg of C₁₂ linear fatty alcohol

380 mg of C₁₄ linear fatty alcohol

190 mg of C₁₂ saturated linear fatty acid

190 mg of C₁₄ saturated linear fatty acid

190 mg of C₁₆ saturated linear fatty acid

230 mg of C₁₈ monounsaturated linear fatty acid

150 mg of C₂₂ monounsaturated linear fatty acid

and is heated at 50° C. until a homogeneous solution is obtained, at which point 0.5 ml of water and 120 mg of immobilized Lipozyme RM IM enzyme are added. The mixture is agitated vigorously twice for 2 minutes and deposited on a 0.5 g lock of hair which is wound up at the bottom of the flask, which is brought to 38° C. for 30 minutes. The lock is subsequently withdrawn and rinsed copiously under the tap at 40° C. It is subsequently washed in two phases with Dop shampoo at 3% (2 ml twice). After rinsing, it is dried under a hairdryer.

5 portions of 1 cm of 5 different hair samples are cut at the middle of the hair, which has been measured beforehand, and are introduced into the intake of a Hewlett-Packard 5890 series II gas chromatograph.

| Number of shampooings | Remanence to Dop-brand shampoo at 3% |
|---|---|
| after treatment | (expressed as μg of wax per g of hair) |
| 1 | 500 |
| 3 | 400 |
| 5 | 250 |
| 7 | 200 |

This experiment shows that after 5 shampooings, at least 50% of the waxes deposited are still fixed on the hair. The products do in fact exhibit remanence for up to 7 shampooings.

### EXAMPLE 2

Remanence Measured for a Triglyceride/alcohol/enzyme Reaction Effected in situ on the Hair:

In a 200 ml wide-necked flask a mixture is formed from

0.4 g of colza oil

0.4 g of coprah oil

0.4 g of palm oil

1 g of dodecanol

1 g of tetradecanol.

The mixture is subsequently heated at 50° C. until a homogeneous solution is obtained and then 1.2 ml of water and 300 mg of immobilized RM IM enzyme are added.

The mixture is agitated vigorously twice for 2 minutes then deposited on a lock of 1.3 g of hair which is wound up at the bottom of the flask. The flask is brought to 38° C. for 30 minutes. The lock is subsequently withdrawn and rinsed copiously under the tap at 40° C. It is subsequently washed twice with Dop-brand shampoo at 3% (2 ml twice). After rinsing, it is dried under a hairdryer.

5 portions of 1 cm of 5 different hair samples are cut at the middle of the hair, which has been measured beforehand, and are introduced into the intake of a Hewlett-Packard 5890 series II gas chromatograph.

| | Remanence to Dop-brand |
|---|---|
| Number of shampooings | shampoo at 3% (expressed as μg of |
| after treatment | fatty acid esters per g of hair) |
| 1 | 3000 |
| 3 | 300 |
| 5 | 200 |
| 7 | 200 |

The products exhibit remanence for up to 7 shampooings.

### EXAMPLE 3

Remanence Measured if the Acid/alcohol/enzyme Solution is Prepared Beforehand

The aim of this experiment is to verify whether the acid/alcohol/enzyme solution is as effective in terms of remanence on the hair if it has been prepared in advance instead of extemporaneously.

In a beaker, a mixture is formed from:

760 mg of C₁₂ alcohol

760 mg of C₁₄ alcohol

380 mg of C₁₂ fatty acid

380 mg of C₁₄ fatty acid

380 mg of C₁₆ fatty acid

500 mg of C₁₈ monounsaturated fatty acid

50 mg of C₂₂ monounsaturated fatty acid

1 ml of water

and 240 mg of Lipozyme RM IM.

The mixture obtained is placed in an oven at 38° C. for 30 minutes.

The remanence to shampooing is studied in the same way as in experiment 1.

The chromatograph of the lotion obtained in the beaker shows that the waxes have formed before they are deposited on the hair. Moreover, after shampooing with Dop at 3%, less than 100 μg of waxes have remained on the hair. And, after 5 washes, there are no longer any detectable remanent waxes.

### EXAMPLE 4

Remanence Measured if the Triglyceride/alcohol/enzyme Solution is Prepared Beforehand

The aim of this experiment is to verify whether the triglyceride/alcohol/enzyme solution is as effective in terms of remanence on the hair if it has been prepared in advance instead of extemporaneously.

In a 200 ml wide-necked flask a mixture is formed from

0.4 g of colza oil

0.4 g of coprah oil

0.4 g of palm oil

1 g of dodecanol

1 g of tetradecanol.

The mixture is subsequently heated at 50° C. until a homogeneous solution is obtained and then 1.2 ml of water and 300 mg of immobilized enzyme RM IM are added.

The mixture is agitated vigorously twice for 2 minutes then placed at 38° C. for 30 minutes in an oven.

The chromatograph of the lotion obtained in the flask shows that the waxes have formed before being deposited on the hair.

The remanence to shampooing is studied in the same way as in experiment 1.

After 1 shampooing there are less than 100 μg of fatty acid esters per gram of hair. After 5 shampooings, the remanent fatty acid esters are at the detection limit.

### EXAMPLE 5

Evaluation of the Action of Lipozyme RM IM on the Deposition of Waxes on the Hair

The same experiment as that described in example 1 was conducted in the presence or absence of immobilized enzyme Lipozyme RM IM in the solution to be deposited on the hair.

The chromatography profiles show that in the absence of Lipozyme RM IM there is no formation of waxes on the hair.

### EXAMPLE 6

Evaluation of the Action of Lipozyme RM IM on the Deposition of Fatty Acid Esters on the Hair

The same experiment as that described in example 2 was conducted in the presence or absence of immobilized enzyme Lipozyme RM IM in the solution to be deposited on the hair.

The chromatography profiles show that in the absence of Lipozyme RM IM there is no formation of fatty acid esters on the hair.

### EXAMPLE 7

Evaluation of the Action of Exogenous Fatty Acids on the Deposition of Waxes on the Hair

The same experiment as that described in example 1 was conducted in the presence or absence of exogenous fatty acids in the solution to be deposited on the hair.

The chromatography profiles show that in the absence of exogenous fatty acids there is no formation of waxes on the hair. Accordingly, the formation of waxes and their remanence on the hair depend on the presence in the solution used of fatty acid, fatty alcohols and exogenous enzymes.

### EXAMPLE 8

Evaluation of the Cosmetic Modifications by Hair Metrology with the Mixture of Fatty Alcohols, Fatty Acids and Enzymes

The comparative evaluation of the cosmetic properties was performed on delipidized and non-delipidized locks of hair with the mixture of fatty alcohols, fatty acids and enzyme by means of a sonometer test. The objective of this test is to quantify the noise from passing a comb through disentangled hair. This noise may be greater or lesser according to the surface condition of the hair, the quality of the ends and the material deposited, even in very small amounts.

A comb is combined with a microphone. The sound captured by this microphone during the passage of the comb through the hair is calibrated by a sonometer whose analogue outlet is connected to a voltage integrator. The result attached corresponds to the sum of the various noises collected in the course of the measurement. Means and standard deviations of the measured noise:

| | | Standard |
|---|---|---|
| Formulas | Mean | deviation |
| Delipidized control | 1546 | 158 |
| Delipidized + enzymatic | 330 | 124 |
| treatment + acids and alcohols | | |
| Non-delipidized controls | 2101 | 242 |
| Non-delipidized + enzymatic | 906 | 206 |
| treat-ment + acids and alcohols | | |

It is clearly demonstrated that the treatment with acids+alcohols and enzyme induces a decrease in the noise generated and hence an improvement in the surface condition of the hair. This effect is obtained both on delipidized natural hair and on non-delipidized natural hair.

### EXAMPLE 9

Evaluation of the Cosmetic Modifications by Hair Metrology with the Mixture of Fatty Alcohols, Triglycerides and Enzymes

The comparative evaluation of the cosmetic properties was performed on delipidized and non-delipidized locks of hair with the mixture of fatty alcohols, triglycerides and enzymes by means of a sonometer test. The objective of this test is to quantify the noise from passing a comb through disentangled hair. This noise may be greater or lesser according to the surface condition of the hair, the quality of the ends and the material deposited, even in very small amounts.

A comb is combined with a microphone. The sound captured by this microphone during the passage of the comb through the hair is calibrated by a sonometer whose analogue outlet is connected to a voltage integrator. The result attached corresponds to the sum of the various noises collected in the course of the measurement. Means and standard deviations of the measured noise:

| | | Standard |
|---|---|---|
| Formulas | Mean | deviation |
| Delipidized control | 1546 | 158 |
| Delipidized + enzymatic | 243 | 103 |
| treatment + oils and alcohols | | |
| Non-delipidized controls | 2101 | 242 |
| Non-delipidized + enzymatic | 1500 | 249 |
| treat-ment + oils and alcohols | | |

It is clearly demonstrated that the treatment with oils (triglycerides)+alcohols and enzyme induces a decrease in the noise generated and hence an improvement of the surface condition of the hair. It is important to note that this effect is obtained both on delipidized natural hair and on non-delipidized natural hair.

1. Cosmetic composition comprising:
a) at least one alcohol of formula R₁—OH, in which R₁ is an optionally substituted, saturated or unsaturated, linear or branched C₃ to C₃₂ alkyl group with or without polyethoxylation,
b) at least one acid of general formula R₂—COOH, in which R₂ is an optionally substituted, saturated or unsaturated, linear or branched C₃ to C₃₁ alkyl group, with the number of carbons in (R₁+R₂) 8, or at least one triglyceride, and
c) at least one exogenous enzyme capable of inducing esterification.

2. Cosmetic composition according to claim 1, comprising:
a) at least one alcohol of formula R₁—OH, in which R₁ is an optionally substituted, saturated or unsaturated, linear or branched C₃ to C₃₂ alkyl group with or without polyethoxylation,
b) at least one acid of general formula R₂—COOH, in which R₂ is an optionally substituted, saturated or unsaturated, linear or branched C₃ to C₃₁ alkyl group, with the number of carbons in (R₁+R₂) 8, and
c) at least one exogenous enzyme capable of inducing esterification.

3. Cosmetic composition according to claim 1, comprising:
a) at least one alcohol of formula R₁—OH, in which R₁ is an optionally substituted, saturated or unsaturated, linear or branched C₃ to C₃₂ alkyl group with or without polyethoxylation,
b) at least one triglyceride, and
c) at least one exogenous enzyme capable of inducing esterification.

4. Cosmetic composition according to any one of claims 1 to 3, characterized in that the enzymes are selected from lipases, esterases and proteases.

5. Cosmetic composition according to any one of claims 1 to 4, characterized in that the enzymes may be in free form or immobilized on a support.

6. Cosmetic composition according to any one of claims 1 to 5, characterized in that the enzymes are immobilized on a support.

7. Cosmetic composition according to any one of claims 1 to 6, characterized in that the amount of enzymes is between 0.0001 and 50% by weight relative to the mixture by weight of alcohols and acids or of alcohols and triglycerides.

8. Cosmetic composition according to any one of claims 1 to 6, characterized in that the amount of enzymes is between 0.1 and 20% by weight relative to the mixture by weight of alcohols and acids or of alcohols and triglycerides.

9. Cosmetic composition according to any one of claims 1 to 2 and 4 to 8, characterized in that the proportion of acids may vary between 1 and 99% relative to the mixture by weight of acids and alcohols.

10. Cosmetic composition according to any one of claims 1 to 2 and 4 to 8, characterized in that the proportion of acids may vary between 30 and 70% relative to the mixture by weight of acids and alcohols.

11. Cosmetic composition according to any one of claims 1 and 3 to 8, characterized in that the proportion of triglycerides may vary between 1 and 99% relative to the mixture by weight of acids and alcohols.

12. Cosmetic composition according to any one of claims 1 and 3 to 8, characterized in that the proportion of triglycerides may vary between 30 and 70% relative to the mixture by weight of acids and alcohols.

13. Cosmetic composition according to any one of claims 1 to 12, characterized in that the proportion of alcohols may vary between 1 and 99% relative to the mixture by weight of acids and alcohols.

14. Cosmetic composition according to any one of claims 1 to 12, characterized in that the proportion of alcohols may vary between 30 and 70% relative to the mixture by weight of acids and alcohols.

15. Cosmetic composition according to any one of claims 1 to 14, comprising adjuvants selected from carbonic oils, silicone oils, fluoro oils, gelling agents, thickeners, emollients, softeners, antioxidants, opacifiers, stabilizers, ionic, non-ionic or amphoteric polymers, vitamins, perfumes, preservatives, dyes, pigments or any other adjuvant which is commonly used in cosmetology and is compatible with the enzyme or enzymes that are used.

16. Cosmetic composition according to any one of claims 1 to 15, characterized in that it is in the form of a gel, cream or lotion.

17. Device comprising a plurality of compartments, characterized in that it includes at least two compartments each containing one or two of the following components:
one or more alcohols of formula R₁—OH in which R₁ is an optionally substituted, saturated or unsaturated, linear or branched C₃ to C₃₂ alkyl group with or without polyethoxylation,
one or more acids of general formula R₂—COOH in which R₂ is an optionally substituted, saturated or unsaturated, linear or branched C₃ to C₃₁ alkyl group, with the number of carbons in (R₁+R₂) 8, or one or more triglycerides,
one or more exogenous enzymes capable of inducing esterification,
the entirety of the device comprising at least one alcohol, an acid or a triglyceride and an enzyme.

18. Use of a composition according to any one of claims 1 to 17 to impart a soft and shiny appearance to the hair fibre.

19. Method of cosmetically treating the hair to impart a soft and shiny appearance to the hair fibre, consisting in mixing on the hair the elements of the device according to claim 17 and then immediately applying the resulting composition to the keratin fibres, leaving it to act for from 1 to 60 minutes at a temperature between 20° C. and 65° C., and optionally carrying out rinsing with water.

20. Method of cosmetically conditioning the hair according to claim 19, wherein the temperature is between 30° C. and 65° C.

21. Method according to one of claims 19 and 20, characterized in that the treatment which allows the soft and shiny appearance to be imparted comprises an in situ enzymatic reaction.
